(19) United States
(12) Patent Application Publication
(10) Pub. No.: US 2021/0290661 A1
(43) Pub. Date: Sep. 23, 2021

(54) IMMUNE MODIFYING PARTICLES FOR THE TREATMENT OF CANCER

(71) Applicant: onCour Pharma, Inc., Northbrook, IL (US)

(72) Inventor: John PUISIS, Northbrook, IL (US)

(21) Appl. No.: 17/264,522

(22) PCT Filed: Jul. 31, 2019
(86) PCT No.: PCT/US2019/044454

§ 371 Date: Jan. 29, 2021

Related U.S. Application Data
(60) Provisional application No. 62/712,604, filed on Jul. 31, 2018.

(51) Int. Cl. A61K 31/765; A61K 33/26; A61K 33/30; A61K 33/242; A61K 33/38; A61K 33/24; A61K 31/745; A61K 39/395; A61K 33/44; A61K 9/14; A61K 45/06; A61K 39/07; A61P 35/00

(52) U.S. Cl. CPC A61K 31/765; A61K 33/26; A61K 33/30; A61K 33/242; A61K 33/38; A61K 33/24; A61K 2039/523; A61K 39/3955; A61K 33/44; A61K 9/14; A61K 45/06; A61K 39/07; A61P 35/00; A61K 31/745

(57) ABSTRACT

The present disclosure provides methods of treating cancer and proliferative diseases using immune-modifying particles in combination with cancer therapeutics, such as checkpoint inhibitors or biologic agents, to modify the activity of myeloid-derived suppressor cells (MDSCs), tumor-associated macrophages (TAMs), neutrophils, monocytes, and/or tumor-associated stroma associated with promoting tumor growth and metastasis.

### FIELD OF THE DISCLOSURE

The present disclosure relates, in general, to methods of treating cancer and proliferative diseases using immune-modifying particles in combination with cancer therapeutics, such as checkpoint modulators, small molecules or biologic agents, by altering myeloid-derived suppressor cells (MDSCs), tumor-associated macrophages (TAMs), neutrophils, and monocytes in the tumor microenvironment.

### BACKGROUND

Distinct myeloid-derived cell populations are now widely recognized as being part of the tumor microenvironment. These cells include monocytes, tumor associated macrophages (TAMs), myeloid-derived suppressor cells (MDSCs) and dendritic cells (Kumar et al., Trends Immunol., 37(3):208-220 (2016); Richards et al., Cancer Microenviron., 6(2):179-91 (2013)). A significant body of work suggests that these cells are actively recruited to tumor sites and sites of metastasis where they can be altered by cues from the local milieu to promote an immunosuppressive microenvironment (Kumar et al., Trends Immunol., 37(3):208-220 (2016); Kitamura et al., Front Immunol., 8:2004 (2018); Kitamura et al., J Exp Med., 212(7):1043-59 (2015)). The immunosuppressive activity of these cells has been shown to promote tumor growth, proliferation, angiogenesis, metastasis, and tumor immune evasion. Furthermore, presence of MDSCs and TAMs in the tumor microenvironment (TME) is correlated with poor disease outcomes (Gabrilovich et al., Nat Rev Immunol., 12(4):253-68 (2012); Ouzounova et al., Nat Comm., 8:14979 (2017); Marvel et al J Clin Invest., 125(9): 3356-64 (2015)).

In addition to immune cells, the tumor stroma plays a role in shaping the tumor microenvironment and influencing tumor growth and progression. Cellular and molecular constituents of the tumor-associated stroma including fibroblasts, mesenchymal stromal cells, adipocytes, endothelium, and the extracellular matrix (ECM) have all been shown to contribute to tumorigenesis (Valkenburg et al., Nat Rev Clin Oncol., 15(6):366-381 (2018)). Tissue-resident and bone marrow mesenchymal stem cell (MSC)-derived cancer associated fibroblasts (CAFs) have been shown to secrete growth factors and proteins that alter anti-tumor immune responses and promote tumor growth and metastasis (Valkenburg et al., Nat Rev Clin Oncol., 15(6):366-381 (2018); Shiga et al., Cancers, 7, 2443-2548 (2015)). Similarly, ECM proteins, mesenchymal stromal cells, endothelial cells, and adipocytes have been reported to dampen anti-tumor immunity and facilitate tumor progression (Lu et al., J Cell Biol., 196(4):395-406 (2012); Kumar et al., Cancer Cell 32(5):654-668.e5 (2017); Quante et al., Cancer Cell., 19, 257-272 (2011); Park et al., Endocr Rev., 32(4):550-70 (2011); Young et al., Cancer Immunol Immunother., 61(10):1609-16 (2012); Hida et al., Int J Mol Sci., 19(5):1272 (2018)).

While significant progress has been made in developing novel anti-cancer therapeutics, the efficacy of these therapies has shown limited promise due to the fact that they target tumors but not immune suppressive factors in the tumor microenvironment (TME) that inhibit anti-tumor immune function and promote tumor progression.

Signaling through immune checkpoint receptors (e.g., Programmed cell death protein 1 (PD-1) and CTLA-4) and their ligands (e.g., PD-L1) regulates the activity of cytotoxic T-cells and has been shown to play a critical role in modulating inflammatory immune responses. Importantly, numerous tumor types are known to hijack the PD-1/PD-L1 and CTLA-4 immune checkpoint signaling pathways in order to evade the T-cell-mediated anti-tumor immune responses. Thus, targeting of immune checkpoint signaling pathways, using specific inhibitors like monoclonal antibodies, has emerged as an attractive frontline treatment option in numerous cancers (Alsaab et al., Front Pharmacol 8:561 (2017)). However, the presence of myeloid-derived suppressor cells (MDSCs) and Tumor-associated macrophages (TAMs) in the tumor microenvironment along with the tumor-associated stroma have been shown to dampen the anti-tumor efficacy of immune checkpoint inhibitors like anti-PD1 monoclonal antibodies (Weber et al., Front Immunol., 9:1310, (2018); Highfill et al., Sci Transl Med., 6(237):237ra67 (2014); Zhao et al., Cancer Immunol Res., 6(12):1459-1471 (2016); Wang et al., Nat Commun., 9(1):3503 (2018)).

Immune modifying Particles (IMPs) are negatively charged nanoparticles that have immunomodulatory properties (See e.g. US Patent Publication Nos. US20150010631; US 20130323319).

### SUMMARY

The present disclosure provides a method of treating cancer and proliferative disease comprising administering immune modifying particles free from antigens or other bioactive agents capable of suppressing monocytes and other phagocytic cells in a subject, in combination with a cancer therapy. Without being bound by theory, it is hypothesized that IMPs alter immunosuppressive monocyte-derived cells in the tumor microenvironment, which in turn will boost the efficacy of other cancer therapies when administered to the subject. It is suggested herein that combination therapies using IMPs and cancer therapeutics that can target both tumor cells and overcome the immunosuppressive tumor microenvironment by targeting MDSCs, TAMs, neutrophils, other monocyte-derived cells, and the tumor-associated stroma may be able to provide enhanced therapeutic benefit compared to monotherapy with cancer therapeutics, such as immune checkpoint inhibitors.

In various embodiments, the disclosure provides a method of treating cancer in a subject comprising administering to the subject negatively charged particles in combination with a cancer therapeutic, wherein said particle is free from attached peptide or antigenic moieties or other bioactive agents and wherein the administering alters the population of myeloid-derived suppressor cells (MDSCs), tumor-associated macrophages (TAMs), neutrophils, monocytes, dendritic cells, and/or stroma at the tumor site.

In various embodiments, the disclosure provides a method of treating cancer in a subject comprising administering to the subject negatively charged particles in combination with a cancer therapeutic, wherein said particle is free from attached peptide or antigenic moieties or other bioactive agents and wherein the administering alters the tumor-associated stroma.

In various embodiments, the disclosure provides a method of treating cancer in a subject comprising administering to the subject negatively charged particles in combination with a cancer therapeutic, wherein said particle is free from attached peptide or antigenic moieties or other bioactive agents and wherein the administering alters stromal connective tissue, fibroblasts, endothelium, adipose tissue, extracellular matrix, pericytes, cancer stem cells, mesenchymal stem cells, and/or mesenchymal stromal cells.

In various embodiments, the disclosure provides a method of treating cancer in a subject comprising administering to the subject negatively charged particles in combination with a cancer therapeutic, wherein said particle is free from attached peptide or antigenic moieties or other bioactive agents and wherein the administering reduces tumor size and/or tumor growth. In various embodiments, the administering induces tumor cell death, apoptosis, and/or necrosis via direct particle uptake by tumor cells.

In various embodiments, the disclosure provides a method of treating cancer in a subject comprising administering to the subject negatively charged particles in combination with a cancer therapeutic, wherein said particle is free from attached peptide or antigenic moieties or other bioactive agents and wherein the administering regulates the anti-tumor immune response.

In various embodiments, the disclosure provides a method of treating cancer in a subject comprising administering to the subject negatively charged particles in combination with a cancer therapeutic, wherein said particle is free from attached peptide or antigenic moieties or other bioactive agents and wherein the administering alters or modulates the tumor-specific immune response.

In various embodiments, the disclosure provides a method of treating a proliferative disease in a subject comprising administering to the subject negatively charged particles in combination with a cancer therapeutic, wherein said particle is free from attached peptide or antigenic moieties or other bioactive agents and wherein the administering alters the population of myeloid-derived suppressor cells (MDSCs), tumor-associated macrophages (TAMs), dendritic cells, neutrophils and/or monocytes at the tumor site.

In various embodiments, the disclosure provides a method of treating a proliferative disease in a subject comprising administering to the subject negatively charged particles in combination with a cancer therapeutic, wherein said particle is free from attached peptide or antigenic moieties or other bioactive agents and wherein the administering alters the tumor-associated stroma.

In various embodiments, the disclosure provides a method of treating a proliferative disease in a subject comprising administering to the subject negatively charged particles in combination with a cancer therapeutic, wherein said particle is free from attached peptide or antigenic moieties or other bioactive agents and wherein the administering alters stromal connective tissue, fibroblasts, endothelium, adipose tissue, extracellular matrix, pericytes, mesenchymal stem cells, and/or mesenchymal stromal cells.

In various embodiments, the disclosure provides a method of treating a proliferative disease in a subject comprising administering to the subject negatively charged particles in combination with a cancer therapeutic, wherein said particle is free from attached peptide or antigenic moieties or other bioactive agents and wherein the administering reduces tumor size and/or tumor growth.

In various embodiments, the disclosure provides a method of treating a proliferative disease in a subject comprising administering to the subject negatively charged particles in combination with a cancer therapeutic, wherein said particle is free from attached peptide or antigenic moieties or other bioactive agents and wherein the administering regulates an anti-tumor immune response.

In various embodiments, the disclosure provides a method of treating a proliferative disease in a subject comprising administering to the subject negatively charged particles in combination with a cancer therapeutic, wherein said particle is free from attached peptide or antigenic moieties or other bioactive agents and wherein the administering regulates a tumor-specific immune response.

In various embodiments, the disclosure provides a method of treating a cancer or a proliferative disorder in a subject comprising administering to the subject negatively charged particles in combination with a cancer therapeutic, wherein said particle is free from attached peptide or antigenic moieties or other bioactive agents and wherein the administering regulates cancer stem cells.

In various embodiments, the negatively charged particles are polyglycolic acid (PGA) particles, polylactic acid (PLA) particles, polystyrene particles, poly (lactic-co-glycolic acid) (PLGA) particles, diamond particles, or iron, zinc, cadmium, gold, or silver particles, or combinations thereof.

In some embodiments, the negatively charged particles are poly (lactic-co-glycolic acid) (PLGA) particles. In various embodiments, the particle comprises about 50:50, about 80:20 to about 100:0 polylactic acid:polyglycolic acid or from about 50:50, about 80:20 to about 100:0 polyglycolic acid:polylactic acid. In various embodiments, the particle comprises 50:50 polylactic acid:polyglycolic acid. In various embodiments, the particle comprises polylactic acid:polyglycolic acid from about 99:1 to about 1:99, including all values and ranges that lie in between these values.

In various embodiments the particles are surface functionalized. In various embodiments, surface functionalization is achieved by carboxylation. In further embodiments, surface functionalization is achieved by the addition of targeting agents. In some embodiments, the targeting agent comprises polypeptides, antibodies, nucleic acids, lipids, small-molecules, carbohydrates, and surfactants. In various embodiments, surface functionalized nanoparticles are targeted preferentially to monocytes, neutrophils, macrophages, dendritic cells, T-cells, B-cells, NK cells, NK T-cells, fibroblasts, cancer associated fibroblasts, endothelial cells, adipocytes, pericytes, endothelium, vasculature, lymphatic vessels, tumor-associated vasculature, mesenchymal stromal cells, mesenchymal stem cells, and/or extracellular matrix.

In various embodiments, the particles have a zeta potential between −100 mV and −1 mV. In various embodiments, the particles have a zeta potential between −80 mV and −30 mV. In some embodiments, the zeta potential of the particle is from about −100 mV to about −40 mV, from about −75 mV to about −40 mV, from about −70 mV to about −30 mV, from about −60 mV to about −35 mV, or from about −50 mV to about −40 mV. In various embodiments, the zeta potential is about −30 mV, −35 mV, −40 mV, −45 mV, −50 mV, −55 mV, −60 mV, −65 mV, −70 mV, −75 mV, −80 mV, −85 mV, −90 mV, −95 mV or −100 mV, including all values and subranges that lie between these values.

In various embodiments, the diameter of the negatively charged particle is between 0.1 μm to 10 μm. In various embodiments, the particle has an average diameter of between about 0.2 μm and about 2 μm; between about 0.3 μm to about 5 μm; between about 0.5 μm to about 3 μm; or between about 0.5 μm to about 1 μm. In some embodiments, the particle has a diameter of about 100 to 1500 nm, about 200 to 2000 nm, about 100 to 10000 nm, about 300 to 1000 nm, about 400 to 800 nm, or about 200 to 700 nm. In various embodiments, the particle has an average diameter of about 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm, including all values and subranges that lie between these values. In some embodiments, the diameter of the negatively charged particle is between 400 nm to 800 nm.

In various embodiments, the subject has a cancer selected from the group consisting of brain cancer, skin cancer, eye cancer, breast cancer, prostate cancer, pancreatic cancer, lung cancer, esophageal cancer, head and neck cancer, cervical cancer, liver cancer, colon cancer, colorectal cancer, rectal cancer, bone cancer, uterine cancer, ovarian cancer, bladder cancer, stomach cancer, oral cancer, thyroid cancer, kidney cancer, testicular cancer, leukemia, lymphoma and mesothelioma. Additional cancers contemplated by the methods are disclosed in the Detailed Description.

In various embodiments, the cancer therapeutic is a chemotherapeutic selected from the group consisting of growth inhibitors, DNA-replication inhibitors, kinase inhibitors, signaling cascade inhibitors, angiogenesis inhibitors, metabolic inhibitors, amino acid synthesis inhibitors, selective inhibitors of oncogenic proteins, inhibitors of metastasis, inhibitors of anti-apoptosis factors, apoptosis inducers, nucleoside signaling inhibitors, enzyme inhibitors and DNA-damaging agents.

In various embodiments, the cancer therapeutic comprises one or more biologic agents selected from the group consisting of cytokines, angiogenesis inhibitors, enzymes, immune checkpoint modulators and monoclonal antibodies.

In various embodiments, cytokines are selected form the group consisting of transforming growth factors, tumor necrosis factor, interferons and interleukins. Exemplary cytokines include, but are not limited to, IFN-alpha, IFN-beta, IFN-gamma, IL-1, IL-2, IL-4, IL-5, IL-6, IL-7, IL-10, IL-11, IL-12, IL-13, IL-15, IL-17, IL-18, IL-21, members of the transforming growth factor beta superfamily, including TGF-β1, TGF-β2 and TGF-β3, tumor necrosis factor alpha, Granulocyte colony-stimulating factor (G-CSF), and Granulocyte macrophage colony-stimulating factor (GM-CSF).

In various embodiments the monoclonal antibodies are mono-specific, bi-specific, tri-specific or bispecific T-cell engaging (BiTE) antibodies.

In various embodiments the monoclonal antibodies are immune cell co-stimulatory molecule agonists that induce an anti-tumor immune response. Exemplary co-stimulatory molecules include, but are not limited to, ICOS (Inducible T cell Co-stimulator) (CD278), OX40 (CD134), 41 BB, GITR (Glucocorticoid-induced Tumor Necrosis Factor Receptor), CD40 and CD27.

In various embodiments, the immune checkpoint modulators target Programmed cell death protein 1 (PD-1), Programmed cell death protein ligand-1 (PD-L1), cytotoxic T-lymphocyte-associated protein 4 (CTLA-4), T-cell Immunoglobulin and mucin-domain containing-3 (TIM-3), Lymphocyte-activation Gene 3 (LAG-3) and/or TIGIT (T cell immunoreceptor with Ig and ITIM domains). In various embodiments, the immune checkpoint modulator is an antibody selected from the group consisting of ipilimumab, tremelimumab, pembrolizumab, nivolumab, atezolizumab, avelumab, cemiplimab and durvalumab.

In various embodiments, monoclonal antibodies useful in the methods are selected from the group comprising Alemtuzumab, Bevacizumab, Brentuximab, Cetuximab, Denosumab, Ibritumomab, Trastuzumab, Panitumumab, Pertuzumab, and Rituximab. In various embodiments, monoclonal antibodies useful in the methods target receptor tyrosine kinase, EGFR, VEGF, VEGFR, PDGF, PDGFR, TGF-β, TGF-β-LAP, SIRP-α, CD47, CD39, CD73, and fibroblast activating protein (FAP).

In various embodiments, the cancer therapeutic comprises an enzyme. In various embodiments, the cancer therapeutic comprises an enzyme that targets T-cells, B-cells, APCs, macrophages, dendritic cells, monocytes, MDSCs, TAMs, neutrophils, other monocyte-derived cells, tumor-associated stroma, cancer stem cells, mesenchymal stem cells, extracellular matrix, and amino acids. In various embodiments, the cancer therapeutic comprises an enzyme selected from the group comprising asparaginase, kynurininase, L-arginine deiminase, L-methionine-γ-lyase, one or more amino acid degrading enzymes, and one or more nucleoside degrading enzymes.

In various embodiments, the cancer therapeutic comprises one or more cell-based therapies selected from the group consisting of adoptive cell transfer, tumor-infiltrating leukocyte therapy, chimeric antigen receptor T-cell therapy (CAR-T), NK-cell therapy and stem cell therapy.

In various embodiments the cell-based therapy is the adoptive transfer of autologous patient-derived cells. In various embodiments the cell-based therapy is the adoptive transfer of allogenic donor-derived cells.

In various embodiments, the cell-based therapy is the transfer of universal donor-derived or induced pluripotent stem cell-derived cells that are not patient specific and amenable to long-term storage. Such therapies are also referred to as ‘off-the-shelf’ therapies.

In various embodiments, the cancer therapeutic is a hormone therapy. In various embodiments, the cancer therapeutic comprises one or more antibody-drug conjugates. In various embodiments, the cancer therapeutic comprises one or more cancer vaccines. In various embodiments, the cancer vaccine is a protein, polypeptide, nucleic acid vaccine and/or a dendritic cell vaccine.

In various embodiments, the cancer therapeutic is an immunotherapy selected from the group comprising oncolytic virus, bacteria, oncolytic bacteria or other bacterial compositions, Bacillus Calmette-Guerin (BCG), a microbiome modulator, and/or a toll-like receptor (TLR) agonist. In various embodiments, the TLR agonist is a TLR3, TLR4, TLR5, TLR7, TLR8, TLR9, TLR10, TLR11, TLR12, and/or TLR13 agonist. In various embodiments, the TLR agonist is derived from virus, bacteria and/or made synthetically. In various embodiments, the immunotherapy is a STING pathway modulator.

In various embodiments, the cancer therapeutic comprises a viral or a bacterial vector. In various embodiments, the viral vector is selected from the group comprising adenovirus, adeno-associated virus (AAV), herpes simplex virus, lentivirus, retrovirus, alphavirus, flavivirus, rhabdovirus, measles virus, Newcastle disease virus, poxvirus, vaccinia virus, modified Ankara virus, vesicular stomatitis virus, picornavirus, tobacco mosaic virus, potato virus x, comovirus or cucumber mosaic virus. In various embodiments, the virus is an oncolytic virus. In various embodiments the virus is a chimeric virus, a synthetic virus, a mosaic virus or a pseudotyped virus.

Additional cancer therapeutics contemplated for use in the methods are set out in the Detailed Description.

In various embodiments, the particle and/or the cancer therapeutic is administered twice weekly, once weekly, once every two weeks, once every three weeks, once every 4 weeks, once every two months, once every three months, once every 6 months or once per year.

In various embodiments, the particle and/or the cancer therapeutic is administered intravenously, orally, nasally, intramuscularly, ocularly, transdermally, or subcutaneously.

In various embodiments, the subject is a mammal. In various embodiments, the subject is human.

In various embodiments, the administration improves one or more symptoms of the cancer or proliferative disorder. In various embodiments, the one or more symptoms are selected from the group consisting of tumor size or tumor burden in the subject, tumor metastasis, and levels of inflammatory cells in the tumor or in the tumor microenvironment. In various embodiments, the administration reduces the tumor size or tumor burden by 10%, 20%, 30% or more. In various embodiments, the administration alters monocytes, macrophages, granulocytes, dendritic cells and/or neutrophils at the tumor.

In various embodiments, the particle is formulated in a composition comprising a pharmaceutical acceptable carrier, diluent or excipient. In various embodiments, the cancer therapeutic is formulated in a composition comprising a pharmaceutical acceptable carrier, diluent or excipient. In various embodiments, the particle and cancer therapeutic can be formulated in the same composition or in separate compositions.

Also contemplated is a composition comprising any of the foregoing particles or cancer therapeutic compositions of the disclosure, or use thereof in preparation of a medicament, for treatment of any of the disorders described herein associated with inflammation and cancer and/or proliferative diseases.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the disclosure. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1 sets out an example treatment schedule for administration of IMP and checkpoint inhibitor anti-PD-1 antibody.

FIG. 2 shows that IMP plus anti-PD-1 combination therapy exhibits improved reduction in tumor growth compared to single therapy treatment in LLC cells.

FIG. 3A-3B shows the anti-tumor efficacy of IMPs when administered alone or in combination with anti-PD1 checkpoint inhibitor antibody. Mice were implanted with MC38 tumor cells and treated with either saline control, IMPs, anti-PD1, or IMPs+anti-PD1 after palpable tumor formation. (FIG. 3A) Compared to control treatment, treatment with IMPs inhibited tumor growth. IMP efficacy was comparable to anti-PD1 treatment. Treatment with IMPs+anti-PD1 demonstrated synergy with an enhanced inhibition of tumor growth. (FIG. 3B) Compared to control treatment, treatment with IMPs prolonged survival. IMP survival efficacy was superior to anti-PD1. Treatment with IMPs+anti-PD1 demonstrated synergy with a prolonged survival compared to control and respective monotherapy.

### DETAILED DESCRIPTION

IMPs present an attractive opportunity to specifically target immunosuppressive monocytes in circulation and prevent them from trafficking to tumor sites where their immunosuppressive activities promote tumor growth, proliferation and metastasis. Combining disruption/alteration of the immune suppressive tumor microenvironment by IMP therapy with other anti-cancer therapeutics, like anti-PD1 monoclonal antibodies, is expected to provide significant benefit over monotherapies that target only tumors.

### Definitions

Each publication, patent application, patent, and other reference cited herein is incorporated by reference in its entirety to the extent that it is not inconsistent with the present disclosure.

It is noted here that as used in this specification and the appended claims, the singular forms “a,” “an,” and “the” include plural reference unless the context clearly dictates otherwise.

“Particle” as used herein refers to any non-tissue derived composition of matter, it may be a sphere or sphere-like entity, bead, or liposome. The term “particle”, the term “immune modifying particle”, and the term “bead” may be used interchangeably depending on the context. Additionally, the term “particle” may be used to encompass beads and spheres.

“Surface-functionalized” as used herein refers to introduction of chemical functional groups to a surface of a particle. Surface functionalized particles may be prepared by free-radical copolymerization of hydrophobic monomers with carboxylic acids, phosphates, hydroxyls, sulfonates, phosphonates, and amine or ammonium groups, as well as other functional groups. General methods of making surface functionalized nanoparticles are described in, for example, Froimowicz et al., Curr Org. Chem 17:900-912, 2013.

“Biodegradable as used herein refers to a particle comprising a polymer that may undergo degradation, for example, by a result of functional groups reacting with the water in the solution. The term “degradation” as used herein refers to becoming soluble, either by reduction of molecular weight or by conversion of hydrophobic groups to hydrophilic groups. Biodegradable particles do not persist for long times in the body, and the time for complete degradation can be controlled. Biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include diamond, PLA, PGA, polypropylene sulfide, and PLGA polymers, as well as metals such as iron (Fe), zinc (Zn), cadmium (Cd), gold or silver. Biocompatible but non-biodegradable materials may also be used in the particles described herein. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

“Negatively charged particle” as used herein refers to particles which have been modified to possess a net surface charge that is less than zero.

Zeta potential is the charge that develops at the interface between a solid surface and its liquid medium. “Negative zeta potential” refers to a particle having a zeta potential of the particle surface as represented in milliVolts (mV), and measured by an instrument known in the field to calculate zeta potential, e.g., a NanoBrook ZetaPlus zeta potential analyzer or Malvern Zetasizer.

“Carboxylated particles” or “carboxylated beads” or “carboxylated spheres” includes any particle that has been modified or surface functionalized to add one or more carboxyl group onto the particle surface. In some embodiments the addition of the carboxyl group enhances phagocyte/monocyte uptake of the particles from circulation, for instance through the interaction with scavenger receptors such as MARCO. Carboxylation of the particles can be achieved using any compound which adds carboxyl groups, including, but not limited to, Poly (ethylene-maleic anhydride) (PEMA).

The term “regulate” or “alter” as used herein refers to modification, modulation or alteration of an immune response at the tumor site or in the tumor microenvironment. Examples of modifying or altering an immune response include, but are not limited to, decreasing immunosuppressive cell numbers or activity at the tumor site or in the tumor microenvironment, reducing immunosuppressive immune cell infiltration at the tumor site or in the tumor microenvironment, increasing pro-inflammatory immune-cell numbers at the tumor site or tumor microenvironment, increasing pro-inflammatory immune-cell infiltration at the tumor site or tumor microenvironment, increasing pro-inflammatory immune-cell activity or function at the tumor site or tumor microenvironment, increasing anti-tumor immune-cell numbers at the tumor site or tumor microenvironment, increasing anti-tumor immune-cell infiltration at the tumor site or in the tumor microenvironment, increasing the anti-tumor activity or function of immune cells at the tumor site or in the tumor microenvironment, and/or killing of cells associated with immunosuppression.

The term “tumor microenvironment” (TME) as used herein refers to cells, molecules, and blood vessels that surround and feed a tumor cell (National Cancer Institute Dictionary of Cancer Terms). The tumor microenvironment includes immune cells, such as bone-marrow derived inflammatory cells, myelo-monocytic cells, myeloid derived suppressor cells, tumor associated-macrophages, dendritic cells, and lymphocytes, stroma, fibroblasts, signaling molecules and the extracellular matrix (ECM) (Joyce et al., Science 348:74-80, 2015).

“Subject” as used herein refers to a human or non-human animal, including a mammal or a primate, that is administered a particle as described herein. Subjects can include animals such as dogs, cats, rats, mice, rabbits, horses, pigs, sheep, cattle, and humans and other primates.

The term “therapeutically effective amount” is used herein to indicate the amount of target-specific composition of the disclosure that is effective to ameliorate or lessen one or more symptoms or signs of the disease or disorder being treated.

The terms “treat”, “treated”, “treating” and “treatment”, as used with respect to methods herein refer to eliminating, reducing, suppressing or ameliorating, either temporarily or permanently, either partially or completely, one or more clinical symptom, manifestation or progression of an event, disease or condition. Such treating need not be absolute to be useful.

### Checkpoint Modulators

Programmed cell death protein 1 (PD-1), also known as cluster of differentiation 279 (CD279), is a cell surface co-inhibitory receptor expressed on activated T cells, B cells and macrophages, and is a component of immune checkpoint blockade (Shinohara et al., Genomics., 23(3):704, (1994); Francisco et al., Immunol Rev., 236: 219, (2010)). PD-1 limits the activity of T cells upon interaction with its two ligands PD-L1 (also known as B7-H1; CD274) and PD-L2 (B7-DC; CD273) (Postow et al., J Clin Oncol., 33: 9, (2015)). Interaction of PD-1 with PD-L1 and PD-L2, reduces T-cell proliferation, cytokine production, and cytotoxic activity (Freeman G J et al., J Exp Med., 192:1027-34, (2000); Brown J A et al., J Immunol., 170:1257-66, (2003)).

Two monoclonal antibodies have been approved by the U.S. Food and Drug Administration (FDA) for the inhibition of PD-1 immunotherapy. Pembrolizumab (KEYTRUDA®, Merck Sharp & Dohme Corp.) is approved for use in metastatic melanoma, and nivolumab (Opdivo®, Bristol-Myers Squibb) is approved for use in metastatic melanoma and metastatic squamous non-small cell lung cancer (NSCLC). Both of these antibodies bind to the PD-1 receptor and block its interaction with its ligands, PD-L1 and PD-L2. In various embodiments, the anti-PD-1 antibody inhibits or blocks binding of the PD-1 receptor to one or both of its ligands, PD-L1 and PD-L2.

Additional antibodies to PD-1 have been described in U.S. Pat. Nos. 8,735,553; 8,617,546; 8,008,449; 8,741,295; 8,552,154; 8,354,509; 8,779,105; 7,563,869; 8,287,856; 8,927,697; 8,088,905; 7,595,048; 8,168,179; 6,808,710; 7,943,743; 8,246,955; and 8,217,149.

Inhibitors of PD-L1 have also been shown to be effective at inhibiting solid tumors in bladder cancer, head and neck cancer, and gastrointestinal cancers (Herbst R S et al., J Clin Oncol., 31: 3000 (2013); Heery C R et al., J Clin Oncol., 32: 5s, 3064 (2014); Powles T et al., J Clin Oncol, 32: 5s, 5011(2014); Segal N H et al., J Clin Oncol., 32: 5s, 3002 (2014)).

CTLA-4 (cytotoxic T-lymphocyte-associated protein 4) (CD152) is a protein receptor that is constitutively expressed in regulatory T cells but only upregulated in conventional T cells after activation. CTLA-4 is a member of the CTLA-4/CD28: B7-1/B7-2 co-stimulatory pathway of T cell signaling and APC activation (Grosso et al., Cancer Immun. 13:5, 2013) and functions as a negative regulator of T cell activation by binding to B7-1 or B7-2 to attenuate T cell responses. CTLA-4 is hypothesized to function as an immune checkpoint. Antibodies specific for CTLA-4 include tremelimumab, and ipilimumab (YERVOY®), which has been approved for the treatment of melanoma.

LAG-3 (Lymphocyte-activation gene 3)(CD223) is a cell-surface receptor that is expressed on a number of immune cells including activated T-cells. LAG-3 is a negative regulator of CD8⁺ T-cells and its deficiency is associated with enhanced CD8⁺ T-cell proliferation (Workman et al. J Immunol 174:688-695 (2005)). Importantly, LAG-3 is co-expressed with PD-1 on tumor infiltrating lymphocytes and exhausted T-cells in both pre-clinical tumor models and cancer patient samples and has been shown to promote tumor immune evasion (Andrews et al., Immunol Rev., 276(1):80-96 (2017); Le Mercier et al. Front Immunol., 6:418 (2015); Woo et al. Cancer Res., 15; 72(4):917-27 (2011); Zhou et al. Oncoimmunology 7(7):e1448332 (2018)). LAG-3 inhibition has shown promise as a therapeutic strategy in pre-clinical models (Grosso et al. J Clin Invest., 117(11):3383-92 (2007); Woo et al. Cancer Res., 15; 72(4):917-27 (2011)) and. inhibition of LAG-3 using monoclonal antibodies alone or in combination with other checkpoint inhibitor antibodies such as Nivolumab® and Pembrolizumab® are currently being evaluated in clinical trials for numerous cancers.

TIM-3 (T-cell Immunoglobulin and mucin-domain containing-3) is a transmembrane protein found to be expressed on terminally differentiated and activated T-cells and is responsible for inhibiting T-cell responses and the expression of inflammatory cytokines such as IFN-γ. TIM-3 is co-expressed with PD-1 on tumor infiltrating lymphocytes and its expression is correlated with the T-cell exhaustion and suppression of T-cell responses (Linhares et al., Front Immunol., 9:1909 (2018); Das et al. Immunol Rev. 276(1):97-111 (2017)). High levels of TIM-3 expression on CD8⁺ T-cells is associated with tumor immune evasion and is correlated with poor prognosis for cancer patients (Anderson et al. Immunity 17; 44(5):989-1004 (2016); Das et al. Immunol Rev. 276(1):97-111 (2017)). Moreover, TIM-3 upregulation in PD-1 expressing tumor infiltrating lymphocytes has been shown to mediate resistance to anti-PD-1 therapeutics (Koyama et al., Nat Commun., 7:10501 (2016)). In line with these observations, monoclonal antibody mediated co-blockade of TIM-3 along with anti-PD-1 has shown promising anti-tumor effects in several pre-clinical tumor models (Ngiow et al., Cancer Res., 3540-3551 (2011); Anderson et al. Immunity 17; 44(5):989-1004 (2016)) and several clinical trials evaluating the efficacy of anti-TIM-3 monoclonal antibodies alone or in combination with checkpoint inhibitor antibodies are currently underway.

### Immune-Modifying Particles

In some embodiments, the present disclosure provides for use of a particle with a negative zeta potential and free of associated antigens, peptides or other bioactive materials in the treatment methods. In various embodiments, the particle is a surface functionalized particle.

Particles can be formed from a wide range of materials. The particle is preferably composed of a material suitable for biological use. For example, particles may be composed of glass, silica, polyesters of hydroxy carboxylic acids, polyanhydrides of dicarboxylic acids, or copolymers of hydroxy carboxylic acids and dicarboxylic acids and biocompatible metals. In various embodiments, the particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, particles can be quantum dots, or composed of quantum dots, such as quantum dot polystyrene particles (Joumaa et al. (2006) Langmuir 22: 1810-6). Particles including mixtures of ester and anhydride bonds (e.g., copolymers of glycolic and sebacic acid) may also be employed. For example, particles may comprise materials including polyglycolic acid polymers (PGA), polylactic acid polymers (PLA), polysebacic acid polymers (PSA), poly(lactic-co-glycolic) acid copolymers (PLGA or PLG; the terms are interchangeable), [rho]oly(lactic-co-sebacic) acid copolymers (PLSA), poly(glycolic-co-sebacic) acid copolymers (PGSA), polypropylene sulfide polymers, poly(caprolactone), chitosan, etc. Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include diamond, PLA, PGA, polypropylene sulfide, and PLGA polymers, as well as metals such as iron (Fe), zinc (Zn), cadmium (Cd), gold (Au) or silver (Ag). Biocompatible but non-biodegradable materials may also be used in the particles described herein. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

The particles of the disclosure can be manufactured by any means known in the art. Exemplary methods of manufacturing particles include, but are not limited to, microemulsion polymerization, interfacial polymerization, precipitation polymerization, emulsion evaporation, emulsion diffusion, solvent displacement, and salting out (Astete and Sabliov, J. Biomater. Sci. Polymer Edn., 17:247-289(2006)). Methods of making particles contemplated herein are disclosed in U.S. Pat. No. 9,616,113 and International Patent Publication WO/2017/143346. Manipulation of the manufacturing process for PLGA particles can control particle properties (e.g. size, size distribution, zeta potential, morphology, hydrophobicity/hydrophilicity, polypeptide entrapment, etc.). The size of the particle is influenced by a number of factors including, but not limited to, the concentration of polymer, e.g., PLGA, the solvent used in the manufacture of the particle, the nature of the organic phase, the surfactants used in manufacturing, the viscosity of the continuous and discontinuous phase, the nature of the solvent used, the temperature of the water used, sonication, evaporation rate, additives, shear stress, sterilization, and the nature of any encapsulated antigen or polypeptide.

In various embodiments, the particle comprises polymers, copolymers, dendrimers, diamond nanoparticle, polystyrene nanoparticles or metals. It various embodiments, it is contemplated that the particle comprises polyglycolic acid polymers (PGA), polylactic acid (PLA), polystyrene, copolymers of PGA and PLA (poly(lactide-co-glycolide) (PLGA)), diamond (PLGA), a liposome, PEG, cyclodextran, or metals, such as iron (Fe), zinc (Zn), cadmium (Cd), gold (Au) or silver (Ag).

In various embodiments, the particle is a co-polymer having a molar ratio from about 50:50, about 80:20 to about 100:0 polylactic acid:polyglycolic acid or from about 50:50, about 80:20 to about 100:0 polyglycolic acid:polylactic acid. In some embodiments, the particle is a poly(lactic-co-glycolic acid) particle. In various embodiments, the particle comprises 50:50 polylactic acid:polyglycolic acid. In various embodiments, the particle comprises polylactic acid:polyglycolic acid from about 99:1 to about 1:99, including all values and ranges that lie in between these values.

In some embodiments, the zeta potential of the particle is from about −100 mV to about −1 mV. In some embodiments, the zeta potential of the particle is from about −100 mV to about −40 mV, from about −80 mV to about −30 mV, from about −75 mV to about −40 mV, from about −70 mV to about −30 mV, from about −60 mV to about −35 mV, or from about −50 mV to about −40 mV. In various embodiments, the zeta potential is about −30 mV, −35 mV, −40 mV, −45 mV, −50 mV, −55 mV, −60 mV, −65 mV, −70 mV, −75 mV −80 mV, −85 mV, −90 mV, −95 mV or −100 mV, including all values and ranges that lie in between these values.

In some embodiments, the particle has an average diameter of between about 0.1 μm to about 10 μm. In some embodiments, the particle has an average diameter of between 0.2 μm and about 2 μm. In some embodiments, the particle has a diameter of between about 0.3 μm to about 5 μm. In some embodiments, the particle has a diameter of between about 0.5 μm to about 3 μm. In some embodiments, the particle has a diameter of between about 0.5 μm to about 1 μm. In some embodiments, the particle has a diameter of about 100 to 1500 nm, about 200 to 2000 nm, about 100 to 10000 nm, about 300 to 1000 nm, about 400 to 800 nm or about 200 to 700 nm, including all values and ranges that lie in between these values.

To administer particles as described herein to human or other mammals, the particle may be formulated in a sterile composition comprising one or more sterile pharmaceutically acceptable carriers. The phrase “pharmaceutically or pharmacologically acceptable” refer to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like.

Pharmaceutical compositions of the present disclosure containing a particle herein may contain sterile pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present invention. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers. A variety of aqueous carriers are suitable, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

It is contemplated that the particle may further comprise a surfactant. The surfactant can be anionic, cationic, or nonionic. Surfactants in the poloxamer and poloaxamines family are commonly used in particle synthesis. Surfactants that may be used, include, but are not limited to PEG, Tween-80, gelatin, dextran, pluronic L-63, PVA, methylcellulose, lecithin, DMAB and PEMA. Additionally, biodegradable and biocompatible surfactants including, but not limited to, vitamin E TPGS (D-α-tocopheryl polyethylene glycol 1000 succinate). In certain embodiments, two surfactants are used. For example, if the particle is produced by a double emulsion method, the two surfactants can include a hydrophobic surfactant for the first emulsion, and a hydrophobic surfactant for the second emulsion.

Therapeutic formulations of the particle are prepared for storage by mixing the particle having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, succinate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; or metal complexes (e.g., Zn-protein complexes).

Preparations of particles can be stabilized by lyophilization. The addition of a cryoprotectant such as trehalose can decrease aggregation of the particles upon lyophilization. Any suitable lyophilization and reconstitution techniques can be employed. It will be appreciated by those skilled in the art that lyophilization and reconstitution can lead to varying degrees of antibody activity loss and that use levels may have to be adjusted to compensate.

### Methods of Use

Provided herein is a method of treating cancer or a proliferative disorder in a subject comprising administering a negatively charged particle in combination with a cancer therapeutic, wherein said particle is free from attached peptide or antigenic moieties and wherein the administering alters the population of myeloid-derived suppressor cells (MDSCs), tumor-associated macrophages (TAMs), neutrophils, dendritic cells, and/or monocytes at the tumor site.

In various embodiments, the disclosure provides a method of treating a cancer or a proliferative disorder in a subject comprising administering to the subject negatively charged particles as described herein in combination with a cancer therapeutic, wherein the administering alters the tumor-associated stroma, and/or wherein the administering alters stromal connective tissue, fibroblasts, endothelium, adipose tissue, extracellular matrix, pericytes, mesenchymal stem cells, and/or mesenchymal stromal cells, and/or wherein the administering reduces tumor size and/or tumor growth, and/or wherein the administering regulates an anti-tumor immune response, and/or wherein the administering regulates a tumor-specific immune response.

In various embodiments, the disclosure provides a method of treating a cancer or a proliferative disorder in a subject, comprising administering to the subject negatively charged particles as described herein in combination with a cancer therapeutic, wherein the administering regulates cancer stem cells.

Methods useful in determining the effects of therapeutics on immune cells include, but are not limited to, microscopic analyses, histological assays, cytological assays, flow cytometry, polymerase chain reaction (PCR), quantitative polymerase chain reaction (qPCR), RNA sequencing (RNA-seq), single-cell RNA sequencing (scRNA-seq), next-generation sequencing, whole-exome sequencing, epigenetic sequencing, ATAC-seq, microarray analysis, and mass cytometry or CyTOF. Biomarkers that can be used, alone or in combination, for the evaluation of immune cells include cell surface markers and secreted proteins. Exemplary biomarkers, include, but are not limited to, CD45, CD3, CD4, CD8, CD25, CD44, CD134, CD252, CD137, CD79, CD39, FOXP3, PD-1, LAG-3, TIM-1, IFN-γ, Granzyme, Perforin, CD11b, CD11c, Ly6C, Ly6G, CD14, CD16, CD80, MARCO, CD68, CD115, CD204, CD205, CD206, CD163, CD103, CD103c, F4/80, PD-L1, PD-L2, Arginase, iNOS, ROS, TNF-α, TGF-β, MHC-I, MHC-II, NK1.1, NKG2D, CD244, Ki67, CD19, CD20, CCR2, CXCR3, CCR4, CCR5, CCR6, CCR7, CCR10, CCL2, CCL5, Cx3CR1, CCL10, ICOS, CD40, CD40L, IL1α, IL1β, IL2, IL4, IL5, IL6, IL8, IL12, IL15, IL17, IL21, IL22, TCRγ/δ, TCRα/β, STAT3, ROR1c, and RORγt.

Cancer stem cells (CSCs) have been described as a subset of cells found within solid and hematologic tumors that are tumorigenic, and capable of self-renewal, differentiation. Several reports have described the importance of CSCs in the pathogenesis of a variety of tumors, tumor relapse after therapy, and development of therapeutic resistance. A number of cell surface markers can be used to distinguish CSCs within solid and hematologic tumors. CSC markers include, but are not limited to, CD19, CD20, CD24, CD34, CD38, CD44, CD90, CD133, Aldehyde dehydrogenase 1, CEACAM-6/CD66c, BMI-1, Connexin 43/GJA1, DLL4, EpCAM/TROP1, GLI-1, GLI-2, Integrins, PON1, PTEN, ALCAM/CD166, DPPIV/CD26, Lgr5, Musashi-1, A20, ABCG2, CD15, Fractalkine, HIF-2α, L1 CAM, c-MAF, Nestin, Podoplanin, SOX2, CD96, CD117, FLT3, AFP, CD13, CD90, NF2/Merlin, ABCB5, NGFR, Syndecan-1, Endoglin, STRO-1, and PON1.

Multiple diagnostic tools designed to characterize tumors at the cellular and molecular level are FDA-approved and commercially available. Examples of approved diagnostics include FOUNDATIONONE® CDX, FOUNDATIONONE® LIQUID, FOUNDATIONONE 6 HEME, BRACAnalysis CDx, therascreen EGFR RGQ PCR kit, cobase EGFR Mutation Test v2, PD-L1 IHC 22C3 pharmDx, Abbott Real Time IDH1, MRDx BCR-ABL test, VENTANA ALK (D5F3) CDx Assay, Abbott RealTime IDH2, Praxis Extended RAS Panel, Oncomine Dx Target Test, LeukoStrat CDx FLT3 Mutation Assay, Foundation Focus CDxBRCA Assay, Vysis CLL FISH Probe Kit, KIT D816V Mutation Detection, PDGFRB FISH, cobas KRAS Mutation Test, therascreen KRAS RGQ PCR Kit, FerriScan, Dako c-KIT pharmDx, INFORM Her-2/neu, PathVysion HER-2 DNA Probe Kit, SPOT-LIGHT HER2 CISH Kit, Bond Oracle HER2 IHC System, HER2 CISH pharmDx Kit, INFORM HER2 DUAL ISH DNA Probe Cocktail, HercepTest, HER2 FISH pharmDx Kit, THXID BRAF Kit, Vysis ALK Break Apart FISH Probe Kit, cobas 4800 BRAF V600 Mutation Test, VENTANA PD-L1 (SP142) Assay, therascreen FGFR RGQ RT-PCR Kit, and therascreen PIK3CA RGQ PCR Kit.

It is contemplated herein that after treatment with a negatively charged particle described herein, optionally in combination with a cancer therapeutic, the level of one or more of biomarkers increases by an amount in the range of from about 1.1 fold to about 10 fold, e.g., about 1.1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10 fold. After treatment with a surface functionalized particle described herein, the level of one or more of the biomarkers decrease by an amount in the range of from about 1.1 fold to about 10 fold, e.g., about 1.1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10 fold.

Exemplary diseases, conditions or disorders that can be treated using the methods herein include cancers, such as esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non small cell lung cancer (NSCLC), squamous cell non-small cell lung cancer, breast cancer, recurrent metastatic breast cancer, hepatocellular carcinoma, hodgkin's lymphoma, follicular lymphoma, non-hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, richter's syndrome; waldenstrom macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, colorectal cancer, rectal cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent merkel cell carcinoma; stage III merkel cell carcinoma; stage IV merkel cell carcinoma, myelodysplastic syndrome and recurrent mycosis fungoides and Sezary syndrome. In various embodiments, the cancers are selected from brain cancer, skin cancer, eye cancer, breast cancer, prostate cancer, lung cancer, esophageal cancer, head and neck cancer, cervical cancer, liver cancer, bone cancer, uterine cancer, ovarian cancer, bladder cancer, stomach cancer, oral cancer, thyroid cancer, kidney cancer, testicular cancer, leukemia, lymphoma and mesothelioma.

It is contemplated that the methods herein reduce tumor size or tumor burden in the subject, and/or reduce metastasis in the subject. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%, including all values and ranges that lie in between these values.

In various embodiments, the disclosure provides a method of reducing the number of immunosuppressive cells from the tumor site by sequestering them in the spleen and/or liver, and inducing apoptosis in a subject comprising administering to the subject negatively charged particles in combination with a cancer therapeutic.

In various embodiments, the administration of the particles in a subject prevents the accumulation of pathology causing monocytes, macrophages, granulocytes and/or neutrophils at the tumor site or tumor microenvironment.

In various embodiments, the disclosure provides a method of reducing the number of monocytes, macrophages, granulocytes and/or neutrophils at the tumor. In various embodiments, the number of monocytes, macrophages, granulocytes and/or neutrophils at the tumor is reduced by about 2-, 3-, 4-, 5-, 6-, 7-, 8-, 9-, or 10-fold or more.

In various embodiments, the disclosure provides a method of altering the anti-tumor immune response in a subject, comprising administering to a subject negatively charged particles in combination with a cancer therapeutic. In various embodiments, the altering increases levels or activity of anti-tumor APCs, macrophages, dendritic cells, T-cells, B-cells, NK T-cells, and/or NK cells.

In various embodiments, the administration of the particles in a subject alters the activity and/or function of immune cells at the tumor site or tumor microenvironment. In various embodiments, the administration of the particles increases the number of inflammatory immune cells at the tumor site or tumor microenvironment. In various embodiments, the administration of the particles in the subject increases the anti-tumor inflammatory function or activity of immune cells at the tumor site or tumor microenvironment.

In various embodiments, the disclosure provides a method of altering the tumor-associated stroma, comprising administering to a subject negatively charged particles in combination with a cancer therapeutic.

In various embodiments, the administration of the particles in a subject alters fibroblasts, cancer-associated fibroblasts, adipocytes, endothelial cells, pericytes, mesenchymal stromal cells, and/or ECM at the tumor site or tumor-associated stroma.

In various embodiments, the disclosure provides a method of reducing tumor size and/or tumor growth in a subject, comprising administering to a subject negatively charged particles in combination with a cancer therapeutic.

In various embodiments, the disclosure provides a method of altering cancer stem cells and/or mesenchymal stem cells, comprising administering to a subject negatively charged particles in combination with a cancer therapeutic.

In various embodiments, the administration of the particles in a subject induces tumor-cell death, tumor-cell apoptosis, and/or tumor cell-necrosis via direct particle uptake.

### Administration and Dosing

Contemplated herein are methods comprising administering a negatively charged particle as described herein in combination with a cancer therapeutic to treat a subject suffering from cancer or a proliferative disorder.

Methods of the disclosure are performed using any medically-accepted means for introducing a therapeutic directly or indirectly into a mammalian subject, including but not limited to injections, oral ingestion, intranasal, topical, transdermal, parenteral, inhalation spray, vaginal, or rectal administration. The term parenteral as used herein includes subcutaneous, intravenous, intramuscular, intraarticular, intraperitoneal, intrathecal and intracisternal injections, as well as catheter or infusion techniques. In various embodiments, the particle is administered intravenously, but may be administered through other routes of administration such as, but not limited to: intradermal, subcutaneous, epictuaneous, oral, intra-articular, and intrathecal. In various embodiments, the compositions are administered at the site of the tumor.

In various embodiments, the particle is administered at a dose from about 0.1 to about 10 mg/kg. In various embodiments, the particle is administered at a dose of about 0.1 mg/kg, 0.5 mg/kg, 1.0 mg/kg, 2.0 mg/kg, 4.0 mg/kg, 8.0 mg/kg or 10 mg/kg. In various embodiments, the particle is administered at a dose of about 8.0 mg, 80 mg, 320 mg, 640 mg or 800 mg. Also contemplated are values within and between the recited dose endpoints. These concentrations may be administered as a single dosage form or as multiple doses.

It is contemplated that the cancer therapeutic, if a known cancer therapeutic, is administered as directed by the manufacturer and the treating physician. If the particle and cancer therapeutic are to be administered in the same formulation, they can be formulated as described herein.

The amounts of immune modulator or biologic agent cancer therapeutic in a given dosage may vary according to the size of the individual to whom the therapy is being administered as well as the characteristics of the disorder being treated. In exemplary treatments, it may be necessary to administer about 1 mg/day, 5 mg/day, 10 mg/day, 20 mg/day, 50 mg/day, 75 mg/day, 100 mg/day, 150 mg/day, 200 mg/day, 250 mg/day, 500 mg/day or 1000 mg/day. Standard dose-response studies, first in animal models and then in clinical testing, reveals optimal dosages for particular disease states and patient populations.

In various embodiments, a checkpoint modulator, such as a PD-1 antibody, CTLA-4 antibody, or PD-L1 antibody, is administered in a dose range from 0.1 to 15 mg/kg. These concentrations may be administered as a single dosage form or as multiple doses.

The conditions treatable by methods of the present disclosure preferably occur in mammals. Mammals include, for example, humans and other primates, as well as pet or companion animals such as dogs and cats, laboratory animals such as rats, mice and rabbits, and farm animals such as horses, pigs, sheep, and cattle. In various embodiments, the subject is human.

In various embodiments, the particle is administered twice weekly, once weekly, once every two weeks, once every three weeks, once every 4 weeks, once every two months, once every three months, once every 6 months or once per year.

The disclosure further contemplates a sterile pharmaceutical composition comprising a particle as described herein, a cancer therapeutic and a pharmaceutically acceptable carrier.

The disclosure further contemplates a sterile pharmaceutical composition comprising a particle as described herein and a pharmaceutically acceptable carrier.

The disclosure further contemplates a sterile pharmaceutical composition comprising a cancer therapeutic and a pharmaceutically acceptable carrier.

Syringes, e.g., single use or pre-filled syringes, sterile sealed containers, e.g. vials, bottle, vessel, and/or kits or packages comprising any of the foregoing antibodies or compositions, optionally with suitable instructions for use, are also contemplated.

### Combination Therapy

It is contemplated that the particle described herein is administered in combination with a cancer therapeutic to treat cancer of a proliferative disorder. In various embodiments, the cancer therapeutic is a chemotherapeutic, a biologic agent, a cell-based therapy, a hormone therapy, an antibody-drug conjugate, oncolytic virus, or a cancer vaccine. Hormone therapies include Tamoxifen for breast cancer, Zoladex for breast cancer and prostate cancer, Aromatase inhibitors (e.g. anastrazole, letrozole, exemestane). Antibody drug conjugates include Brentuximab vedotin for lymphomas. (anti-CD30 mAB+monomethyl auristatin E), Ado-trastuzumab entansine for breast cancers. (anti-Her2/Neu+maytansinoid) and Inotuzumab Ozagamicin for ALL (anti-CD22+calicheamicin). Oncolytic viruses include Imlygic (Amgen®). Cancer vaccines include Sipuleucel-T for prostate cancer. Several cancer vaccines are in development and include, but are not limited to, proteins, polypeptides, nucleic acids, and dendritic cell vaccines.

In various embodiments, the cancer therapeutic is a chemotherapeutic selected from the group consisting of growth inhibitors, a cytotoxic agent, DNA-replication inhibitors, kinase inhibitors, signaling cascade inhibitors, angiogenesis inhibitors, metabolic inhibitors, amino acid synthesis inhibitors, selective inhibitors of oncogenic proteins, inhibitors of metastasis, inhibitors of anti-apoptosis factors, apoptosis inducers, nucleoside signaling inhibitors, enzyme inhibitors and DNA-damaging agents.

A cytotoxic agent refers to a substance that inhibits or prevents the function of cells and/or causes destruction of cells. The term is intended to include radioactive isotopes (e.g., I131, I125, Y90 and Re186), chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A non-cytotoxic agent may include an agent that can be activated to be cytotoxic.

Chemotherapeutic agents contemplated for use in the methods of the present disclosure include, but are not limited to those listed in Table I:

TABLE I

Alkylating agents
Nitrogen mustards
mechlorethamine
cyclophosphamide
ifosfamide
melphalan
chlorambucil
Nitrosoureas
carmustine (BCNU)
lomustine (CCNU)
semustine (methyl-CCNU)
Ethylenimine/Methyl-melamine
thriethylenemelamine (TEM)
triethylene thiophosphoramide (thiotepa)
hexamethylmelamine (HMM, altretamine)
Alkyl sulfonates
busulfan
Triazines
dacarbazine (DTIC)
Antimetabolites
Folic Acid analogs
methotrexate
Trimetrexate
Pemetrexed (Multi-targeted antifolate)
Pyrimidine analogs
5-fluorouracil
fluorodeoxyuridine
gemcitabine
cytosine arabinoside (AraC, cytarabine)
5-azacytidine
2,2′-difluorodeoxy-cytidine
Purine analogs
6-mercaptopurine
6-thioguanine
azathioprine
2′-deoxycoformycin (pentostatin)
erythrohydroxynonyl-adenine (EHNA)
fludarabine phosphate
2-chlorodeoxyadenosine (cladribine, 2-CdA)
Type I Topoisomerase Inhibitors
camptothecin
topotecan
irinotecan
Biological response modifiers
G-CSF
GM-CSF
Differentiation Agents
retinoic acid derivatives
Hormones and antagonists
Adrenocorticosteroids/antagonists
prednisone and equiv-alents
dexamethasone
ainoglutethimide
Progestins
hydroxyprogesterone caproate
medroxyprogesterone acetate
megestrol acetate
Estrogens
diethylstilbestrol
ethynyl estradiol/equivalents
Antiestrogen
tamoxifen
Androgens
testosterone propionate
fluoxymesterone/equivalents
Antiandrogens
flutamide
gonadotropin-releasing
hormone analogs
leuprolide
Nonsteroidal antiandrogens
flutamide
Natural products
Antimitotic drugs
Taxanes
paclitaxel
Vinca alkaloids
vinblastine (VLB)
vincristine
vinorelbine
Taxotere ® (docetaxel)
estramustine
estramustine phosphate
Epipodophylotoxins
etoposide
teniposide
Antibiotics
actimomycin D
daunomycin (rubido-mycin)
doxorubicin (adria-mycin)
mitoxantroneidarubicin
bleomycin
splicamycin (mithramycin)
mitomycinC
dactinomycin
aphidicolin
Enzymes
L-asparaginase
L-arginase
Radiosensitizers
metronidazole
misonidazole
desmethylmisonidazole
pimonidazole
etanidazole
nimorazole
RSU 1069
EO9
RB 6145
SR4233
nicotinamide
5-bromodeozyuridine
5-iododeoxyuridine
bromodeoxycytidine
Miscellaneous agents
Platinium coordination complexes
cisplatin
Carboplatin
oxaliplatin
Anthracenedione
mitoxantrone
Substituted urea
hydroxyurea
Methylhydrazine derivatives
N-methylhydrazine (MIH)
procarbazine
Adrenocortical suppressant
mitotane (o,p′ - DDD)
ainoglutethimide
Cytokines
interferon (α, β, γ)
interleukin-2
Photosensitizers
hematoporphyrin derivatives
Photofrin ®
benzoporphyrin derivatives
Npe6
tin etioporphyrin (SnET2)
pheoboride-a
bacteriochlorophyll-a
naphthalocyanines
phthalocyanines
zinc phthalocyanines
Radiation
X-ray
ultraviolet light
gamma radiation
visible light
infrared radiation
microwave radiation

It is also contemplated that the cancer therapeutic comprises one or more biologic agents, such as cytokines, angiogenesis inhibitors, immune checkpoint modulators and monoclonal antibodies. Cytokines include interferons (IFN) and interleukins (ILs), such as IFN-alpha, IFN-beta, IFN-gamma, IL-1, IL-2, IL-4, IL-5, IL-6, IL-7, IL-10, IL-11, IL-12, IL-13, IL-15, IL-17, IL-18, IL-21, members of the transforming growth factor beta superfamily, including TGF-β1, TGF-β2 and TGF-β3, tumor necrosis factor alpha, Granulocyte colony-stimulating factor (G-CSF), and Granulocyte macrophage colony-stimulating factor (GM-CSF).

Biologic agents such as immune checkpoint modulators target PD1, PD-L1, CTLA-4, TIMP-3, LAG-3 and/or TIGIT (T cell immunoreceptor with Ig and ITIM domains). In various embodiments, the immune checkpoint modulators are antibodies specific for PD-1, PD-L1, or CTLA-4. Antibodies specific for checkpoint proteins include ipilimumab (YERVOY®, Bristol-Myers Squibb Company), and tremelimumab that bind CTLA-4; antibodies to PD-1 such as Pembrolizumab (KEYTRUDA®, Merck Sharp & Dohme Corp) and nivolumab (OPDIVO®, Bristol-Myers Squibb); and antibodies that target PD-L1 such as Atezolizumab (TECENTRIQ®), Avelumab (BAVENCIO®), and Durvalumab (IMFINZI®) (approved for treatment of urothelial carcinoma and non-small cell lung carcinoma), Cemiplimab (LIBTAYO®) (approved for cutaneous squamous cell carcinoma).

In various embodiments, monoclonal antibodies useful in the methods are selected from the group comprising Alemtuzumab, Bevacizumab, Brentuximab, Cetuximab, Denosumab, Ibritumomab, Trastuzumab, Panitumumab, Pertuzumab, and Rituximab. In various embodiments, monoclonal antibodies useful in the methods target receptor tyrosine kinase, EGFR, VEGF, VEGFR, PDGF, PDGFR, TGF-β, TGF-β-LAP, SIRP-α, CD47, CD39, CD73, and fibroblast activating protein (FAP).

Biologic agents include monoclonal antibodies that are mono-specific, bi-specific, tri-specific or bispecific T-cell engagers (BiTE). Monoclonal antibodies useful in the treatment of cancer include bevacizumab (AVASTIN®, Genentech), an antibody to VEGF-A; erlotinib (TARCEVA®, Genentech and OSI Pharmaceuticals), a tyrosine kinase inhibitor which acts on EGFR, dasatinib (SPRYCEL®, Bristol-Myers Squibb Company), an oral Bcr-Abl tyrosone kinase inhibitor; IL-21; pegylated IFN-α2b; axitinib (INLYTA®, Pfizer, Inc.), a tyrosine kinase inhibitor; and trametinib (MEKINIST®, GlaxoSmithKline), a MEK inhibitor (Philips and Atkins, Int Immunol., 27(1):39-46 (2015) which is incorporated herein by reference). Bispecific antibodies useful to treat cancer are described in Krishnamurthy et al., (Pharmacol Ther. 2018 May; 185:122-134), and Yu et al., (J. Hematol Oncol 2017, 10:155), including Blinatumomab and catumaxomab.

The method also provides that the cancer therapeutic comprises one or more cell-based therapies including adoptive cell transfer, tumor-infiltrating leukocyte therapy, chimeric antigen receptor T-cell therapy, NK-cell therapy and stem cell therapy.

It is contemplated that the cancer therapeutic comprises one or more immunotherapies including oncolytic virus, oncolytic bacteria or other bacterial compositions, microbiome regulators, Bacillus Calmette-Guerin, TLR agonists, a microbiome modulator, STING pathway modulators, and cancer vaccines. The method also provides that the cancer therapeutic comprises a viral or bacterial vector. In various embodiments, the TLR agonist is a TLR3, TLR4, TLR5, TLR7, TLR8, TLR9, TLR10, TLR11, TLR12, and/or TLR13 agonist. In various embodiments, the TLR agonist is derived from virus, bacteria and/or made synthetically.

In various embodiments, the cancer therapeutic comprises an enzyme. In various embodiments, the cancer therapeutic comprises an enzyme that targets T-cells, B-cells, APCs, monocytes, MDSCs, TAMs, neutrophils, other monocyte-derived cells, tumor-associated stroma, cancer stem cells, mesenchymal stem cells, extracellular matrix, and amino acids. In various embodiments, the cancer therapeutic comprises an enzyme selected from the group comprising asparaginase, kynurininase, L-arginine deiminase, L-methionine-γ-lyase, one or more amino acid degrading enzymes, and one or more nucleoside degrading enzymes.

It is contemplated that the particle and the cancer therapeutic can be given concurrently, simultaneously, or sequentially. Concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks.

It is contemplated that the particle and the cancer therapeutic may be given simultaneously, in the same formulation. It is further contemplated that the agents are administered in a separate formulation and administered concurrently, with concurrently referring to agents given within 30 minutes of each other.

In another aspect, the cancer therapeutic is administered prior to administration of the particle composition. Prior administration refers to administration of the cancer therapeutic within the range of one week prior to treatment with the particle, up to 30 minutes before administration of the particle. It is further contemplated that the cancer therapeutic is administered subsequent to administration of the particle composition. Subsequent administration is meant to describe administration from 30 minutes after particle treatment up to one week after administration.

### Kits

As an additional aspect, the disclosure includes kits which comprise one or more compounds or compositions packaged in a manner which facilitates their use to practice methods of the disclosure. In one embodiment, such a kit includes a compound or composition described herein (e.g., a composition comprising a particle alone or in combination with a cancer therapeutic), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the particle, cancer therapeutic, or composition according to a specific route of administration or for practicing a screening assay. Preferably, the kit contains a label that describes use of the inhibitor compositions.

Additional aspects and details of the disclosure will be apparent from the following examples, which are intended to be illustrative rather than limiting.

### EXAMPLES

### Example 1

### Therapeutic Targeting of Tumors Using IMP Therapy in Combination with the Immune Checkpoint Inhibitor Anti-PD-1 Monoclonal Antibody

In order to determine the anti-tumor efficacy of IMP therapy in combination with the PD-1 monoclonal antibody (mAb), orthotopic or syngeneic tumor models will be established using 6-8-week old C57BL/6 mice as described by Kumar et al. (Cancer Cell 32, 654-668, (2017])). For Example, mice will be implanted with LLC (Lewis Lung Carcinoma), MC38 or EL4 (9,10-dimethyl-1,2-benzanthracene-induced murine thymoma) cells by s.c injection and divided randomly into 4 treatment groups each, as described below:

Group 1: Control treatment (n=7)

Group 2: IMPs only (n=10)

Group 3: Anti-PD1 mAb only (n=10)

Group 4: IMPs+Anti-PD1 mAb (n=7)

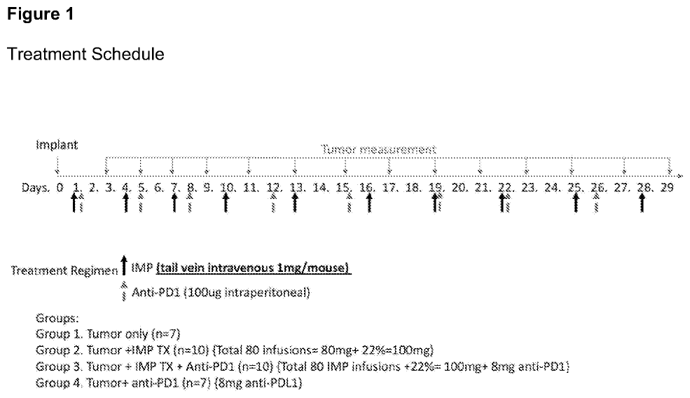

24 hours after s.c. injection with tumor cells, mice are treated with IMPs (1 mg i.v.) and/or anti-PD1 mAb (100 μg i.p.). The following treatment schedule is followed and is illustrated in FIG. 1:

Day 0: s.c. injection with LLC cells or EL4 cells.

| | | IMP | Anti-PD1 mAb |
|---|---|---|---|
| Group | Treatment | Treatment Days | Treatment Days |
| 1 | Saline (Control) | — | — |
| 2 | IMPs only | 1, 4, 7, 10, 13, | — |
| | | 16, 19, 22, 25, 28 | |
| 3 | Anti-PD1 mAb | — | 11, 14, 17, 20, |
| | | | 23, 26, 29 |
| 4 | IMPs + anti-PD1 mAb | 1, 4, 7, 10, 13, | 11, 14, 17, 20, |
| | | 16, 19, 22, 25, 28 | 23, 26, 29 |

Mice receiving control treatment are given saline i.v. and/or i.p instead of IMPs and anti-PD1 mAb. Tumor growth in each treatment group is assessed by measuring tumor area using standard calipers at days 0, 14, 19, 22, 25 and 29.

Mice receiving saline treatment develop large tumors ranging from 500-600 mm² in area by the end of the treatment period, such that euthanasia is warranted. Conversely, combination therapy with both IMPs and anti-PD1 mAb is expected to result in reduction of, to complete abrogation of, tumor growth. Tumors in the combination treatment group may grow to only 5-10 mm² in area, out to 40 days.

Mice treated with IMPs or anti-PD1 mAb alone are expected to show an intermediate effect with significant delays in tumor growth compared to saline-treated mice. It is hypothesized that tumors ranging from 100-150 mm² in area are expected to be seen toward the end of treatment in these groups. Tumors in these treatment groups may grow to 200-250 mm² in area but are not expected to reach the size of tumors seen in saline-treated mice

In an initial experiment, mice were inoculated with LLC tumor cells via s.c injection at Day 0. LLC tumor cells (ATCC®) were maintained in a monolayer culture in DMEM (Corning CellGro®, 10-013-CV) supplemented with 10% FBS (Atlanta Biologicals), 5 nM glutamine, 25 mM HEPES and 1% antibiotics (Invitrogen). A suspension of 0.5×10⁶ LLC cells was prepared and was injected into the right lower flank of 7-10-week-old C57BL/6 mice. At Day 1, mice were randomized into treatment groups and treated according to the following schedule. Tumor growth in each treatment group was assessed by measuring tumor area using standard calipers at days 0, 14, 19, 22, 25 and 29. IMPs were PLGA particles.

| | | IMP | Anti-PD1 mAb |
|---|---|---|---|
| Group | Treatment | Treatment Days | Treatment Days |
| 1 | Saline (Control) | — | — |
| 2 | IMPs only | 1, 4, 7, 10, 13, | — |
| | | 16, 19, 22, 25, 28 | |
| 3 | Anti-PD1 mAb | — | 11, 14, 17, 20, |
| | | | 23, 26, 29 |
| 4 | IMPs + anti-PD1 mAb | 1, 4, 7, 10, 13, | 11, 14, 17, 20, |
| | | 16, 19, 22, 25, 28 | 23, 26, 29 |

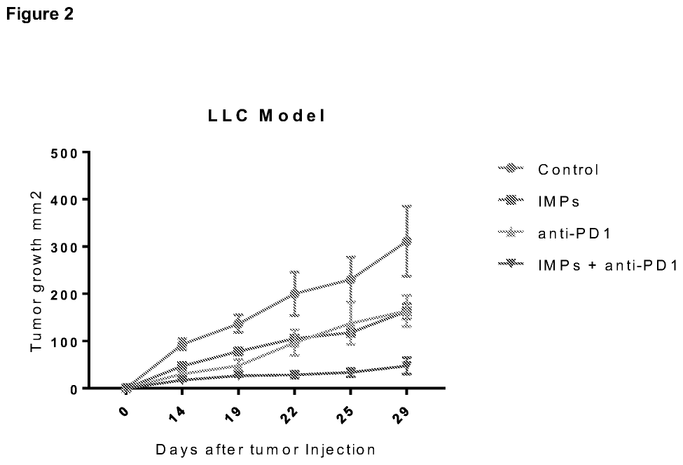

Results are shown in FIG. 2. As expected, saline treated (control) mice developed large tumors by day 29. Monotherapy with anti-PD1 or IMPs alone showed comparable anti-tumor effects with a medial inhibition of tumor growth compared to saline treatment. Combination therapy with IMPs and anti-PD1 mAb resulted in a synergistic effect and enhanced inhibition of tumor growth compared to saline treatment or monotherapy with IMPs or anti-PD-1 mAb.

### Example 2

### Therapeutic Effect of IMPs Administered Alone or in Combination with Anti-PD1

To determine the effects of IMP combination treatment on a syngeneic tumor model, 6-8-week old C57BL/6 mice were implanted with MC38 (colon adenocarcinoma) tumor cells via subcutaneous injection into the flanks. MC38 mouse tumor cells were maintained in a monolayer culture in DMEM supplemented with 10% heat-inactivated fetal bovine serum (FBS) and 2 mM L-glutamine at 37° C. in a tissue-culture incubator with 5% CO₂. After palpable tumor formation (˜100 mm³ in size), animals were randomized into one of four treatment groups as follows:

Group 1: Control treatment (n=10)

Group 2: IMPs (n=10)

Group 3: anti-PD1 (n=10)

Group 4: IMPs+anti-PD1 (n=10)

A single cell suspension of 1×10⁶ 95% viable cells resuspended in 0.1 mL serum-free DMEM was prepared and injected into the right lower flank of the animals. Treatment was started after tumor sizes reached a size of approximately 100 mm³. IMPs (PLGA particles) (1 mg) were administered via intravenous (i.v) injection and anti-PD-1 (100 μg) was administered via intraperitoneal (i.p) injection according to the following treatment schedule:

| | | IMP | Anti-PD1 mAb |
|---|---|---|---|
| | | Treatment Days | Treatment Days |
| | | (After palpable | (After palpable |
| Group | Treatment | tumor formation) | tumor formation) |
| 1 | Control (Saline) | — | — |
| 2 | IMPs | 1, 4, 7, 10, 13, | — |
| | | 16, 19, 22, 25 | |
| 3 | Anti-PD1 mAb | — | 1, 5, 8, 12, 15, |
| | | | 19, 22 |
| 4 | IMPs + anti-PD1 mAb | 1, 4, 7, 10, 13, | 1, 5, 8, 12, 15, |
| | | 16, 19, 22, 25 | 19, 22 |

Tumor growth was evaluated by measuring tumor size in two dimensions using a caliper. Tumor volumes were calculated using the formula V=0.5×a×b², where a and b are the long and short diameters of the tumor, respectively. Tumor sizes were expressed in mm³.

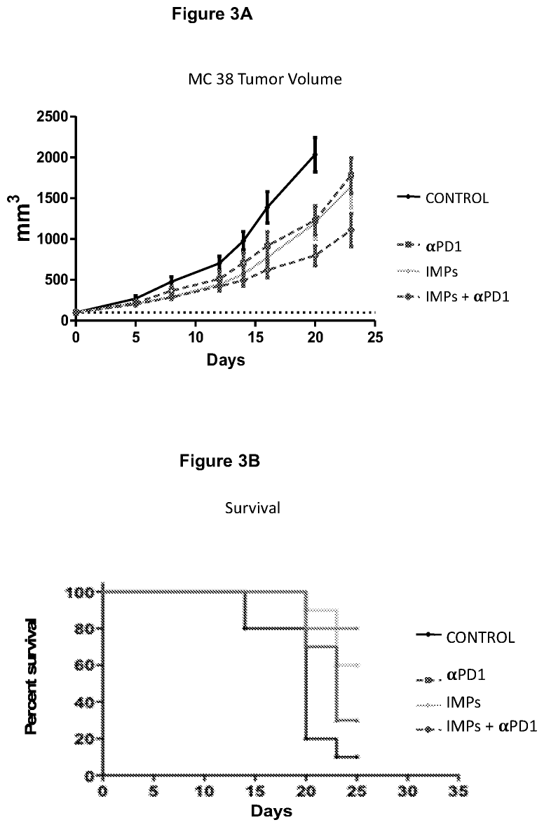

As shown in FIG. 3A, treatment with IMPs led to a strong inhibition of tumor growth compared to control treatment. IMP efficacy was comparable to anti-PD1 treatment. Treatment with IMPs and anti-PD1 in combination demonstrated synergy and led to increased inhibition of tumor growth compared to respective monotherapies. Reflective of its effect on tumor growth, treatment with IMPs led to prolonged survival of MC38 tumor-bearing mice. IMP survival efficacy was superior to anti-PD1 and combination therapy with IMP and anti-PD1 demonstrated a synergistic effect that resulted in enhanced survival when compared to respective monotherapies (FIG. 3B).

Numerous modifications and variations in the disclosure as set forth in the above illustrative examples are expected to occur to those skilled in the art. Consequently, only such limitations as appear in the appended claims should be placed on the disclosure.

What is claimed:

1. A method of treating cancer or a proliferative disease in a subject comprising administering to the subject negatively charged particles in combination with a cancer therapeutic, wherein the particle is free from attached peptide or antigenic moieties or other bioactive agents.

2. The method of claim 1, wherein the administering alters myeloid-derived suppressor cells (MDSCs), tumor-associated macrophages (TAMs), neutrophils and/or monocytes at the tumor site.

3. The method of claim 1 or claim 2, wherein the administering reduces tumor size and/or tumor growth in the subject.

4. The method of any one of claims 1 to 3, wherein the administering regulates the anti-tumor immune response.

5. The method of any one of claims 1 to 4, wherein the administering alters cancer stem cells and/or mesenchymal stem cells.

6. The method of any one of claims 1 to 5, wherein the administering alters the tumor-associated stroma.

7. The method of any one of claims 1 to 6, wherein the administering alters fibroblasts, adipocytes, endothelial cells, mesenchymal stromal cells, and/or the ECM in the tumor-associated stroma.

8. The method of any one of the preceding claims wherein the negatively charged particles are polyglycolic acid polymers (PGA), polylactic acid (PLA), polystyrene particles, or poly (lactic-co-glycolic acid) (PLGA) particles, diamond particles, or iron, zinc, cadmium, gold, or silver particles.

9. The method of any one of the preceding claims, wherein the negatively charged particles are poly (lactic-co-glycolic acid) (PLGA) particles.

10. The method of claim 8 or 9, wherein the particle comprises about 50:50, about 80:20 to about 100:0 polylactic acid:polyglycolic acid or from about 50:50, about 80:20 to about 100:0 polyglycolic acid:polylactic acid.

11. The method of any one of the preceding claims, wherein the particle comprises 50:50 polylactic acid:polyglycolic acid.

12. The method of any one of the preceding claims, wherein the particle is carboxylated.

13. The method of any one of the preceding claims, wherein the particle has a zeta potential between −100 mV and −1 mV.

14. The method of any one of the preceding claims, wherein the particle has a zeta potential between −80 mV and −30 mV.

15. The method of any one of the preceding claims, wherein the diameter of the negatively charged particle is between 0.1 μm to 10 μm.

16. The method of any one of the preceding claims, wherein the diameter of the negatively charged particle is between 400 nm to 800 nm.

17. The method of any one of the preceding claims, wherein the subject has a cancer selected from the group consisting of brain cancer, skin cancer, eye cancer, breast cancer, pancreatic cancer, prostate cancer, lung cancer, esophageal cancer, head and neck cancer, cervical cancer, liver cancer, colon cancer, colorectal cancer, rectal cancer, bone cancer, uterine cancer, ovarian cancer, bladder cancer, stomach cancer, oral cancer, thyroid cancer, kidney cancer, testicular cancer, leukemia, lymphoma and mesothelioma.

18. The method of any one of the preceding claims, wherein the cancer therapeutic is a chemotherapeutic selected from the group consisting of growth inhibitors, DNA-replication inhibitors, kinase inhibitors, signaling cascade inhibitors, angiogenesis inhibitors, metabolic inhibitors, amino acid synthesis inhibitors, selective inhibitors of oncogenic proteins, inhibitors of metastasis, inhibitors of anti-apoptosis factors, apoptosis inducers, enzyme inhibitors, nucleoside signaling inhibitors and DNA-damaging agents.

19. The method of any one of claims 1-17, wherein the cancer therapeutic comprises one or more biologic agents selected from the group consisting of cytokines, enzymes, angiogenesis inhibitors, immune checkpoint modulators and monoclonal antibodies.

20. The method of claim 19, wherein the cytokines are selected from the group consisting of transforming growth factors, tumor necrosis factor, interferons and interleukins.

21. The method of claim 19, wherein the immune checkpoint modulators target Programmed cell death protein 1 (PD1), Programmed cell death protein ligand-1 (PD-L1), cytotoxic T-lymphocyte-associated protein 4 (CTLA-4), T cell Immunoglobulin and Mucin-domain containing-3 (TIM-3), Lymphocyte-activation Gene-3 (LAG-3) and/or TIGIT (T cell immunoreceptor with Ig and ITIM domains).

22. The method of claim 21, wherein the immune checkpoint modulator is an antibody selected from the group consisting of ipilimumab, tremelimumab, pembrolizumab, nivolumab, atezolizumab, avelumab, cemiplimab and durvalumab.

23. The method of claim 19, wherein the monoclonal antibody is mono-specific, bi-specific, tri-specific or bi-specific T-cell engager (BiTE).

24. The method of claim 19, wherein the monoclonal antibody comprises Alemtuzumab, Bevacizumab, Brentuximab, Cetuximab, Denosumab, Ibritumomab, Trastuzumab, Panitumumab, Pertuzumab, and Rituximab.

25. The method of claim 19, wherein the monoclonal antibody targets receptor tyrosine kinase, EGFR, VEGF, VEGFR, PDGF, PDGFR, TGF-β, TGF-β-LAP, SIRP-α, CD47, CD39, CD73 and/or fibroblast activating protein (FAP).

26. The method of any one of claims 1-17, wherein the cancer therapeutic comprises one or more cell-based therapies selected from the group consisting of adoptive cell transfer, tumor-infiltrating leukocyte therapy, chimeric antigen receptor T-cell therapy (CAR-T), NK-cell therapy and stem cell therapy.

27. The method of any one of claims 1-17, wherein the cancer therapeutic is a hormone therapy.

28. The method of any one of claims 1-17, wherein the cancer therapeutic comprises one or more antibody-drug conjugates.

29. The method of any one of claims 1-17, wherein the cancer therapeutic comprises one or more cancer vaccines.

30. The method of any one of claims 1-17, wherein the cancer therapeutic is an immunotherapy comprising oncolytic virus, oncolytic bacteria or other bacterial compositions, Bacillus Calmette-Guerin (BCG), a microbiome modulator, a STING pathway modulator and/or a toll-like receptor (TLR) agonist.

31. The method of any one of the preceding claims wherein the particle and or the cancer therapeutic is administered twice weekly, once weekly, once every two weeks, once every three weeks, once every 4 weeks, once every two months, once every three months, once every 6 months or once per year.

32. The method of any one of the preceding claims, wherein the particles are administered intravenously, orally, nasally, intramuscularly, ocularly, transdermally, or subcutaneously.

33. The method of any one of the preceding claims, wherein the subject is human.

34. The method of any one of the preceding claims wherein the administration improves one or more symptoms of the cancer of proliferative disorder.

35. The method of claim 34, wherein the one or more symptoms are selected from the group consisting of tumor size or tumor burden in the subject, tumor metastasis, and levels of inflammatory cells in the tumor.

36. The method of claim 35, wherein the administration reduces the tumor size or tumor burden by 10%, 20%, 30% or more.

37. The method of claim 34, wherein the administration reduces of the number of monocytes, macrophages, granulocytes and/or neutrophils at the tumor.

38. The method of any one of the preceding claims, wherein the particle is formulated in a composition comprising a pharmaceutical acceptable carrier, diluent or excipient.

39. The method of any one of the preceding claims, wherein the cancer therapeutic is formulated in a composition comprising a pharmaceutical acceptable carrier, diluent or excipient.
